(19) United States
(12) Patent Application Publication
(10) Pub. No.: US 2011/0287546 A1
(43) Pub. Date: Nov. 24, 2011

(54) Automated intracellular manipulation and transfection

(76) Inventor: Allan Yang Wu, (US)

(21) Appl. No.: 12/800,694

(22) Filed: May 20, 2010

(51) Int. Cl. C12N 15/64; C12M 1/00; C12N 15/74

(52) U.S. Cl. 435/455; 435/471; 435/306.1

(57) ABSTRACT

A method and apparatus whereby prokaryotic, eukaryotic and/or mammalian cells may have genetic agents inserted or removed or transferred in an automated and semi-quantitative fashion. The functional unit of the invention is composed of two rectangular plates: a contact plate with circular holes and a carefully aligned base plate with a pleurality of rods, which protrude through the center of the holes of the contact plate. The edges of the two plates are sealed lengthwise to create a space whereby fluid may flow through the proximal and distal openings to induce a negative pressure, vacuum suction, venturi effect at each hole of the contact plate. The rods and base plate may be coated with an electronically magnetizable surface which may attract and hold or repulse and release any magnetically responsive genetic agents.

### BACKGROUND

Somatic nuclear cell transfer is the process of removing the nucleus of a cell and then transplanting it into an empty donor cell. This process has been utilized for mammalian cloning in animal husbandry. Additionally, the procedure has been considered for use in limited therapeutic cloning, in which donor nuclei are transferred into pleuripotent enucleated surrogate cells in an autologous, allogenic or xenographic fashion.

Typically expensive and sensitive micromanipulation equipment is used to perform the procedure under direct microscopic visualization with at least two micromanipulation armatures: one to hold and position the cell and the other to lance the cell and introduce the transferred nucleus. Those skilled in the art of embryology and cellular biology are aware that current equipment and technical skill required to perform nuclear cell transfer are prohibitively expensive, tedious and time consuming.

Transient transfection is also another challenging procedure in cellular and molecular biology. Many different types of genetic manipulating agents may be physically introduced into the cell with direct injection using ballistics, electroporation or transfection agents such as lipofectamine.

For the purposes of this patent genetic agents may be referred to, but not limited to, the following:
RNA (Ribonucleic Acid)
DNA (Deoxiribonucleic Acid)
PNA (Protien Nucleic Acid)
siRNA (RNA intereference agents)
whole chromosomes
partial chromosomes
artificial chromosomes
protein
enzymes
oligonucleotides
antibodies
oligonucleotides
signal transduction agents
toxins
hormones
small molecules
isotopes
cell nuclei
gametes
or any other material that may induce a change in cellular activity at the DNA, RNA, protein and/or signal transduction level.

For the purposes of this patent labels may be referred to, but not limited to, the following:
antibodies
synthetic oligonucleotides
radio-isotopes
DNA
RNA
siRNA
PNA
LNA (locked nucleic acid)
nucleic acids
amino acids
protein
haptens
prions

Transfection using any of the above methods involves extensive manipulation to the cell causing considerable loss in viability. Furthermore, trying to achieve direct quantitative transfection of the genetic manipulating agents is not possible with the above methods.

Yet another challenging field of molecular diagnosis is capture of nuclear proteins. Several nuclear proteins are now used as potential markers for clinical utility. Rapid isolation and diagnosis of the presence of these proteins is time consuming and challenging sometimes due to the harsh denaturing chemical treatments necessary for nuclear protein processing, which destroy delectability of nuclear targets.

Certainly an automated and inexpensive method with the versatility of being capable of performing all the cellular and molecular procedures detailed previously would have considerable utility and biomedical application.

### SUMMARY OF INVENTION

Those familiar with the field of embryology and somatic nuclear cell transfer are aware that a donor nucleus must be removed from a donor cell and transferred into an empty surrogate cell. Essentially, the surrogate cell must be cleared of pre-existing genetic material, namely the nucleus prior to having the donor nucleus transplanted into the surrogate cell. With current technology the process is performed by micromanipulation devices composed of a vacuum pipette tip to hold the surrogate cell and a glass pipette tip to lance the surrogate cell and inject the nucleus into the surrogate cell. The same procedure may be performed by the invention disclosed such that conceptually the lancing element and vacuum pipette is combined into one functional unit.

In simplest terms the functional unit of the patent is composed of two rectangular plates: a contact plate with circular and/or fruste-conical edged holes and a carefully aligned base plate with a pleurality of rods, which protrude through the holes of the contact plate and above the plane of the upper contact plate. The edges of the two plates are sealed lengthwise to create a space whereby fluid may flow between the plates and through the proximal and distal openings to induce a negative pressure, vacuum suction, or venturi force at each hole of the contact plate in a direction perpendicular and toward the plane of the base plate. The proximal and distal openings may also have adjustable flow ports to modulate the flow rate of fluid through the device and effectively change the magnitude of negative pressure, vacuum suction or venturi effect at each hole. The distal opening may also be completely occluded to cause a positive pressure flowing away from the base plate through the holes in the contact plate to push off any cell that was previously seated on the hole. The entire device and process may be carried out within a Petri dish and/or under a microscope. The rods and base plate may be coated with an electronically magnetizable surface. In the ideal embodiment the fruste-conical holes of the base plate are oriented towards the cells to allow a spheroid-type shaped cell to seat itself securely over the hole when a negative pressure is applied through the hole.

For the apparatus to be used for nuclear cell transfer, prior to any contact with the apparatus, donor cells are incubated with magnetized antibodies to the nucleus. After the donor nucleus is significantly coated with magnetic antibodies the donor cells are introduced to the apparatus chamber, wherein they are attracted to the functional unit by a negative pressure vacuum. As the cell is pulled down and seats into the fruste-conical base of the functional unit, the central pole of a piercing rod enters into the cell. The piercing rod is then magnetized and the nucleus is captured on the rod. The negative pressure on the apparatus is reversed such that the donor cell is pushed of the base plate leaving the donor nucleus behind and magnetically immobilized against the piercing rod. The upper chamber and surface of the contact plate is then thoroughly washed so that no residual donor cell cytoplasm or membranes are left behind. Next the enucleated surrogate cells are introduced to the surface of the contact plate. (Note that the surrogate cells may be pretreated in a similar manner, but performed within a similar and separate apparatus to enucleate the cells' nuclei.) The negative vacuum flow is resumed and the surrogate cells are pulled onto the functional unit and pierced by the magnetized rod holding the immobilized donor nucleus. Once the donor cell has seated itself firmly on the rod, the magnetic field of the rod is turned off or polarity reversed to release or repulse the donor nucleus into the cytoplasm of the surrogate cell. The surrogate cell is then released from the base of the base plate by reversing the negative pressure to push off the surrogate cell now with the donor nucleus within it.

Alternatively the same apparatus may be used for transfection or injection of other genetic agents. The procedure is performed in manner similar to somatic nuclear transfer, but instead or donor nuclei being transferred, other magnetized genetic agents are introduced into the surrogate cells. Additionally, the surrogate cells in this alternate process may in fact retain its original nucleus. The magnetic field of the apparatus' magnetizable rods are turned on and attract the magnetized genetic agents, essentially coating the rod. The recipient cell is then introduced to the apparatus chamber and upper surface of the contact plate, and the recipient cells are then seated onto the holes of the contact plate. The magnetic field of the magnetizable rods are then turned off or polarity reversed and the magnetized genetic agent is released or repulsed into the cytoplasm of the cell. The cells are then pushed off the base plate by reversing the flow though the hole to push off the cell. The cell is then transfected with the magnetized genetic agent. The functional unit may be composed of a pleurality of magnetizable rods per hole to provide quantitative or semi-quantitative increments of transfection.

Additionally, the device may also be used for intracytoplasmic sperm injection. Those familiar with the field of animal husbandry and infertility are aware that sperm may be physically injected into embryo with the use of micromanipulators. The same procedure may also be carried out with the disclosed invention, such that the sperm are coated with any suitable magnetizable antibody and immobilized onto the magnetized rod of the apparatus. A one sperm to one rod arrangement is ideal. The embryos are placed onto the contact plate and fluid flow though the proximal and distal ends of the apparatus induce a negative pressure through the holes in a direction towards the base plate creating a mild suction to seat a single embryo over a single rod coated with a single sperm. The magnetic field of the base plate and rod is then turned off and the flow though the distal end of the apparatus (exit port) is blocked to cause flow of fluid through the holes away from the base plate to push off the embryos and complete the intracytoplasmic sperm injection process.

Furthermore, the device may also be used for nuclear molecular diagnosis. Nuclei from cells of interest may be removed for direct staining or labeling with antibodies, FISH, PCR probes, synthetic oligonucleotides or any suitable probe that is visually detectable or measureable. The cells of interest would be prepared in a similar manner such that a magnetized antibody recognizing a nuclear structure is exposed to the cell. The cells are then introduced to the apparatus to allow one cell to be seated onto one rod-hole unit as mentioned previously by modifying fluid flow through the proximal and distal ends (entry and exit ports) of the apparatus. The rod and base plate is then magnetized to capture the nuclei and the cell membrane is pushed off the rod by blocking flow through the distal end (exit port) of the apparatus to expose the nucleus. The nucleus may then undergo exposure to any suitable molecular probe with greater ease, since there is no impeding cytoplasm to occlude binding or visualization of probes. Additionally, the nuclear proteins and structure are left intact since no harsh denaturing lysis buffers are required.

Furthermore, the invention may be constructed from any suitable material and by any suitable means to achieve the functional capabilities outlined above. Those familiar with the field of lithography a nanotechnology are aware that the apparatus of the invention described may be fabricated at the microscopic and/or nanometric level with using many tools and methods commonly available in the field of micro and nano fabrication.

### BRIEF DESCRIPTION OF DRAWINGS

FIG. 1. A conceptual drawing of the smallest functional unit of the invention.

FIG. 2: A conceptual drawing of the smallest functional unit of the invention minus the central magnetic rod in the center. Black arrow representing negative pressure through center of functional unit generated as a result of fluid flow between upper and lower plates represented by large white arrow.

FIG. 3: Conceptual drawing of smallest functional unit in an ideal configuration with a eukaryotic cell.

FIG. 4: Close up view (dotted inset) of magnetized antibody binding to antigens on nuclei and magnetized antibodies in turn being attracted to the magnetic rod. (Magnetic attraction only used for this embodiment.)

FIG. 5: Close up view (dotted box inset) of antibody binding to antigens on nuclei constant region of antibody bound to antibodies immobilized on the magnetizable rod of the apparatus. (No magnetic antibody or magnetic attraction used for this embodiment.)

FIG. 6: Close up view (dotted box inset) of antibody binding to antigens on nuclei constant region of antibody bound to antibodies immobilized on the magnetizable rod of the apparatus in addition to using magnetic antibodies to magnetize the nucleus and allow attraction to the magnetic rod. (Combined use of direct antibody binding and magnetic attraction to magnetizable rod.)

FIG. 7: Conceptual drawing of smallest functional unit after donor cell is pushed off of apparatus and donor nucleus is still left behind on the magnetized rod.

FIG. 8: Conceptual drawing of the smallest functional unit of the apparatus being prepared for intracytoplasmic sperm injection application.

FIG. 9: Conceptual drawing of the smallest functional unit of the apparatus being used for intracytoplasmic sperm injection application.

FIG. 10: Conceptual drawing of the smallest functional unit of the apparatus being prepared for transfection of genetic agents.

FIG. 11: Conceptual drawing of the smallest functional unit of the apparatus being used for transfection of genetic agents.

FIG. 12: Alternate conceptual drawing of the apparatus modified for transfection of genetic agents, but in this case three times the amount of transfection may occur as there are 3 rods.

FIG. 13: 3-fold transfection of genetic agents into the recipient cell.

FIG. 14: Alternate embodiment of genetic agent transfer. using magnetized antibodies to attract bound genetic agents to magnetized magnetizable rod.

FIG. 15: An ideal three dimensional representation of the invention. Note magnetizable rods fit through center of upper plate.

FIG. 16: Longitudinal cross section representation of device.

FIG. 17: Saggital cross section view of device.

FIG. 18: Three dimensional view of patent with entry and exit flow through ports.

### DETAILED DESCRIPTION OF DRAWINGS

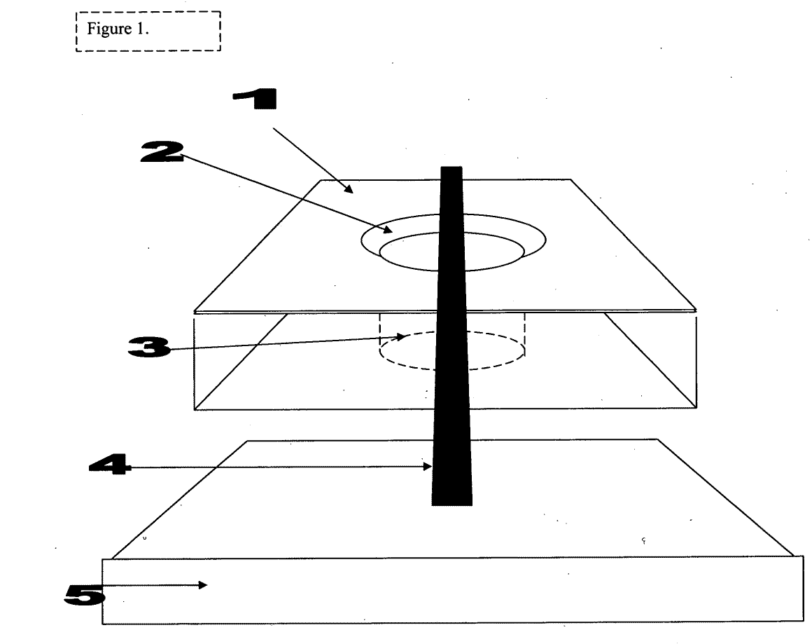

FIG. 1. A conceptual drawing of the smallest functional unit of the invention, whereby the upper contact plate (1) has a central hole (3) through which a magnetizable rod (4) protrudes though and above the surface plane of the upper contact plate (1) and the fruste-conical edge (3). The magnetizable rod (4) is physically attached to the base plate (5) and both may also be electronically magnetizable.

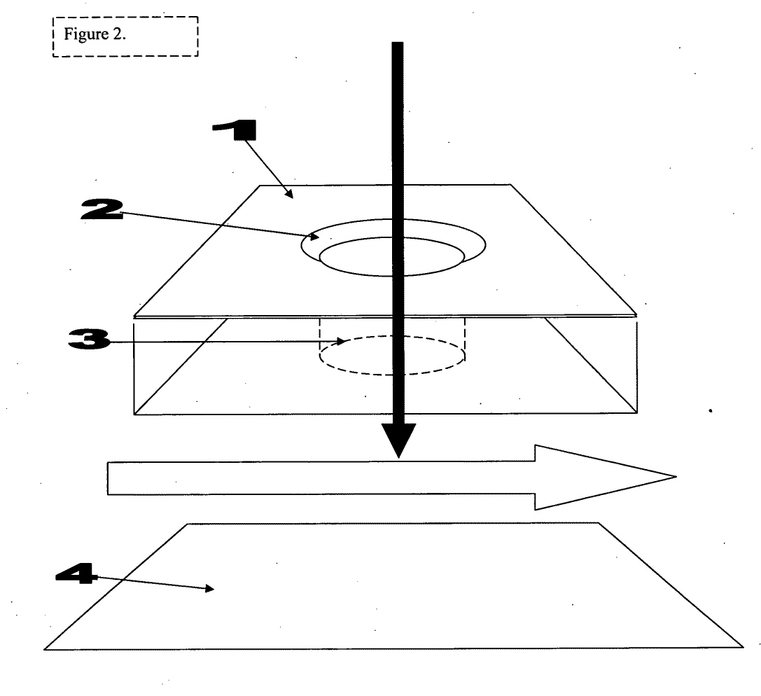

FIG. 2: A conceptual drawing of the smallest functional unit of the invention minus the central magnetic rod through the center hole (3) with fruste-conical edge (3). Black arrow representing negative pressure through center of functional unit is generated as a result of fluid flow (large white arrow) between upper contact (1) and lower base (4) plates.

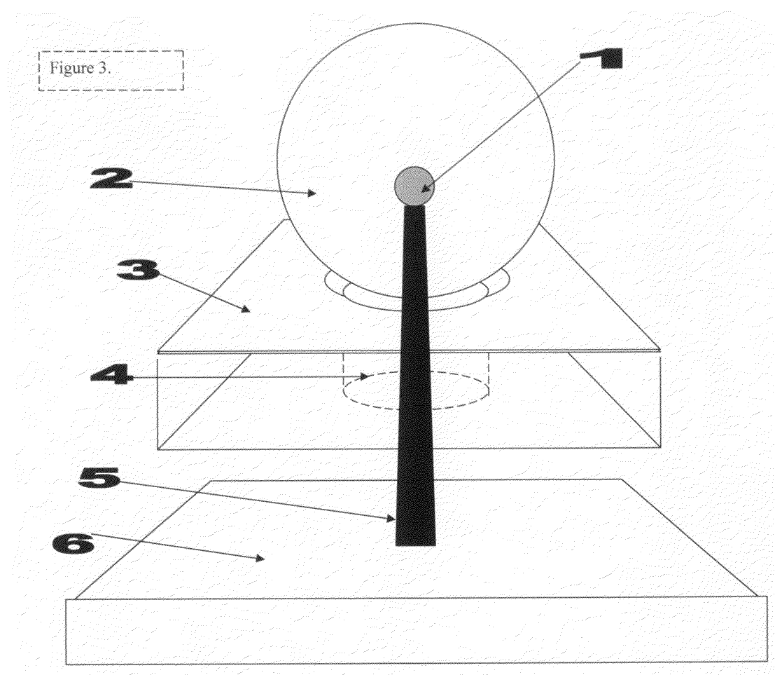

FIG. 3: Conceptual drawing of smallest functional unit in an ideal configuration with a eukaryotic cell (3) and its nucleus (1), sealing the central hole (4) and the magnetizable rod (5) piercing the membrane of the cell (2) and magnetically capturing the nucleus (1). The base plate (6) and magnetizable rod (5) may be physically connected and composed of the same suitable material to allow both to be selectively and electronically magnetizable.

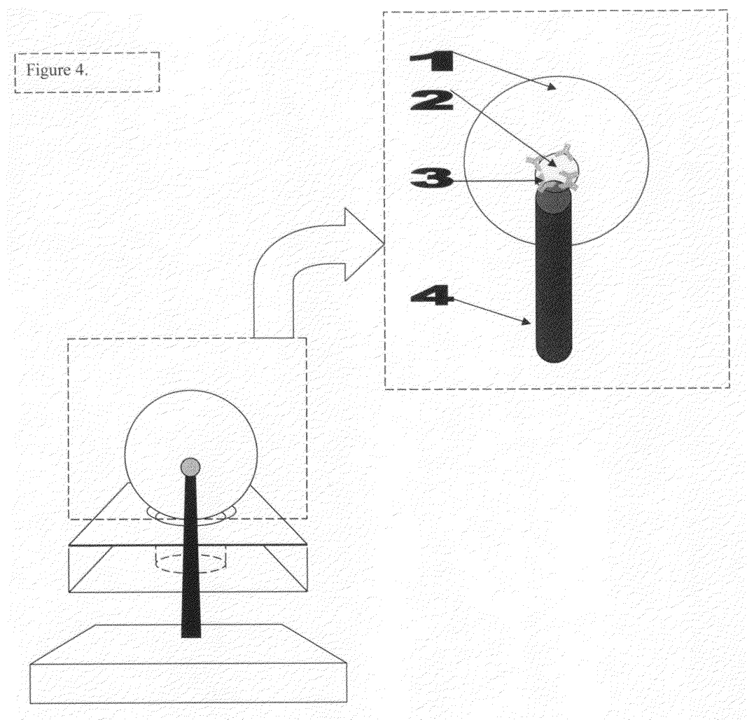

FIG. 4: Close up view (dotted inset) of magnetized antibodies (3) binding to antigens on nuclei (2) and magnetized antibodies (3) in turn being attracted to the magnetized rod (4) with magnetic field turned on (filled in black).

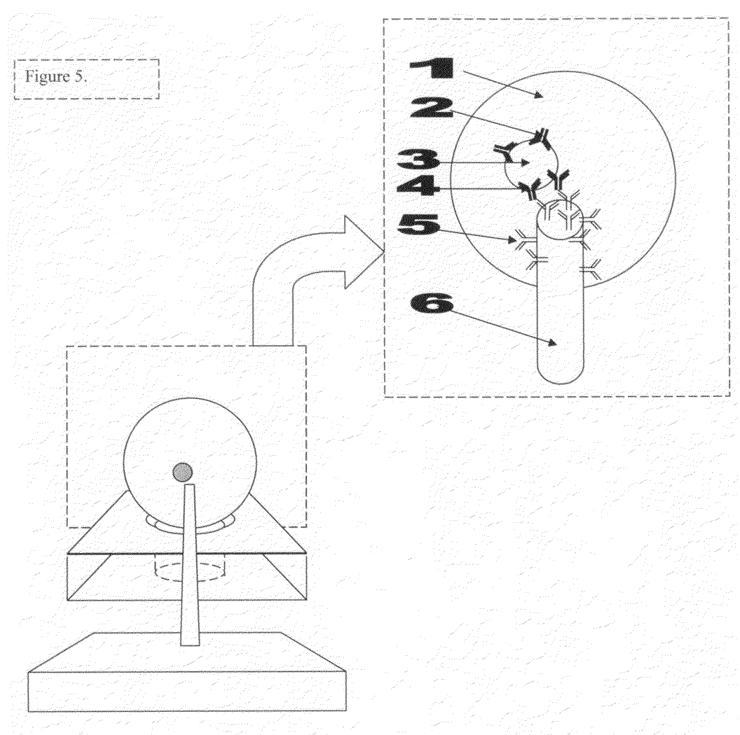

FIG. 5: Close up view (dotted box inset) of antibody (2 and 4) binding to antigens on nuclei (3) of a cell (1) and constant region of antibody (4) bound to antibodies immobilized (5) on the central rod (6) of the apparatus. (No magnetic antibody or magnetic attraction used for this embodiment.)

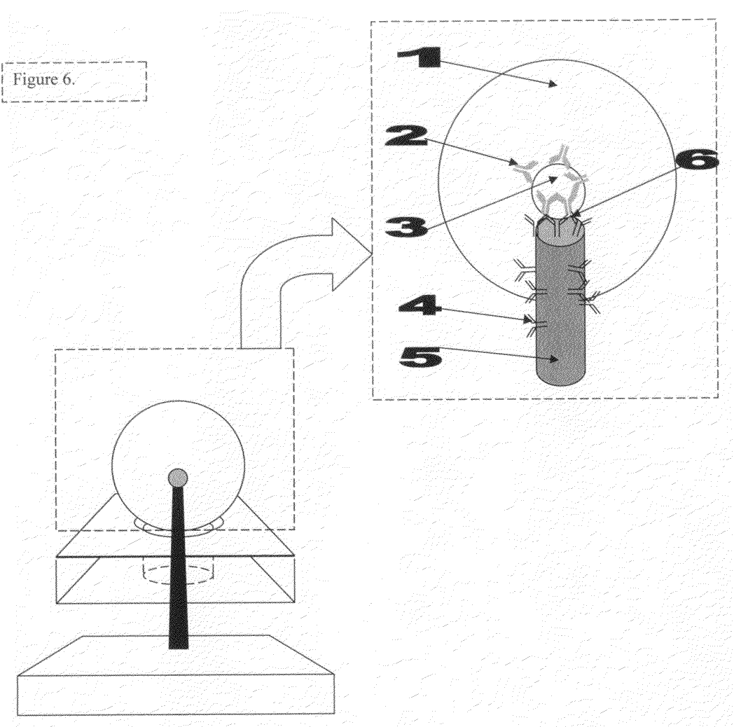

FIG. 6: Close up view (dotted box inset) of antibody binding to antigens on nuclei (3) in a cell (1) with the constant region of antibody (6) bound to antibodies immobilized (4) on the magnetized rod (5) of the apparatus in addition to using magnetic antibodies (2) to magnetize the nucleus (3) and allow attraction to the magnetic rod (5). This approach uses direct antibody binding and magnetic attraction to magnetizable rod.

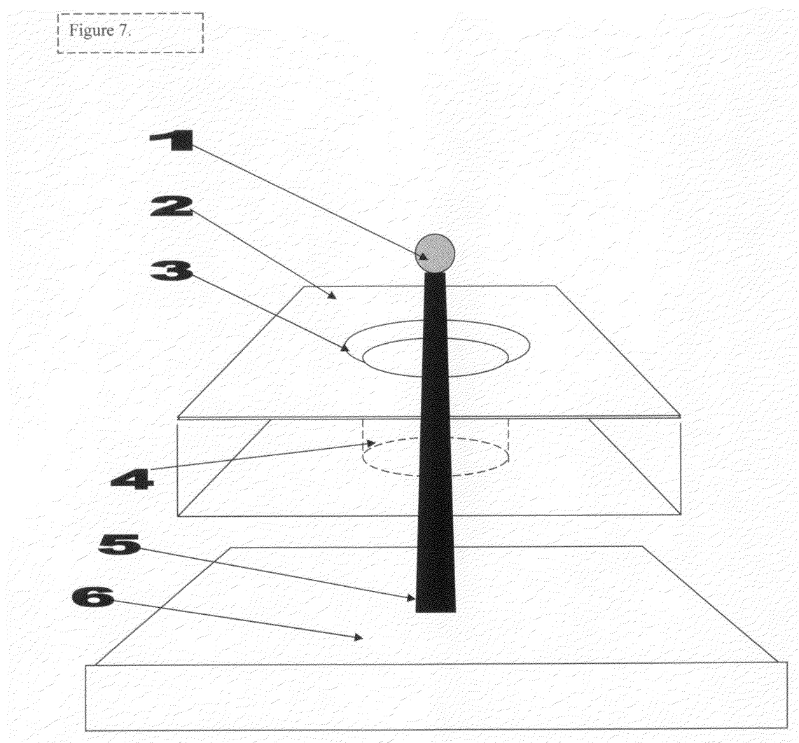

FIG. 7: Conceptual drawing of smallest functional unit after donor cell is pushed off of apparatus and donor nucleus (1) is still left behind on the magnetized rod (5). The upper contact plate (2) has a central hole (4) through which a magnetizable rod (5) protrudes though and above the surface plane of the upper contact plate (2) and the fruste-conical edge (3). The magnetizable rod (4) is physically attached to the base plate (6) and both are currently magnetized as illustrated by the black filled in appearance of the magnetizable rod (5).

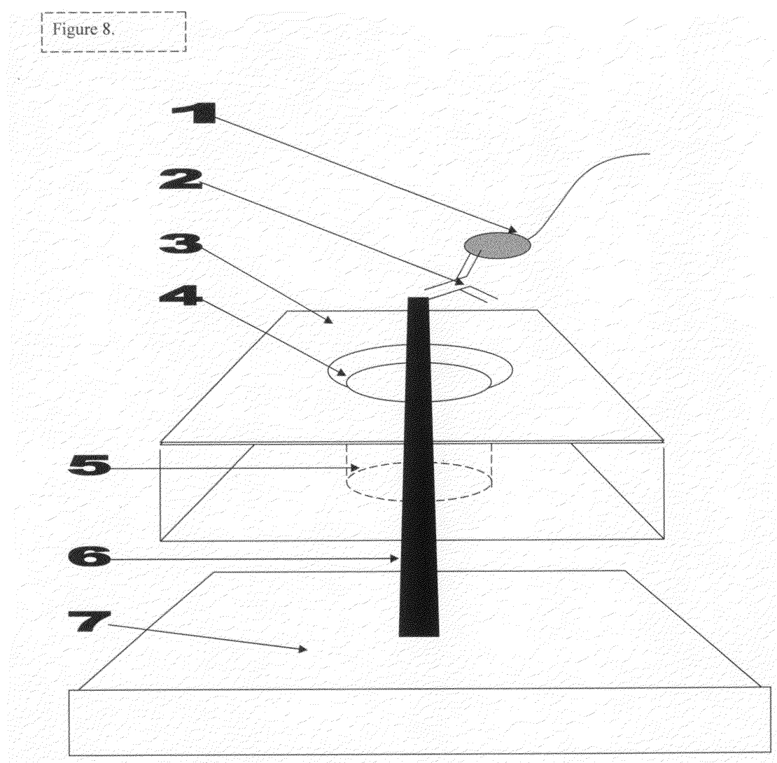

FIG. 8: Conceptual drawing of the smallest functional unit of the apparatus being prepared for intracytoplasmic sperm injection application. The upper contact plate (3) has a central hole (5) through which a magnetizable rod (6) protrudes though and above the surface plane of the upper plate (3) and the fruste-conical edge (4). The magnetizable rod (6) is physically attached to the base plate (7) and both are currently magnetized as illustrated by the black filled in appearance of the magnetizable rod (5), which magnetically immobilizes the magnetized antibody (2) binding to antigens on the sperm (1).

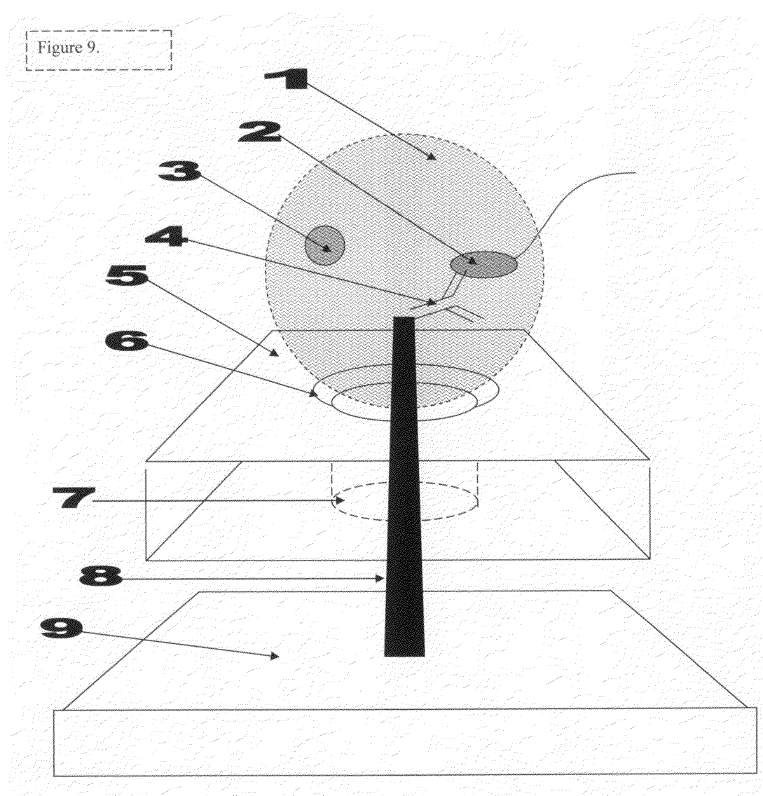

FIG. 9: Conceptual drawing of the smallest functional unit of the apparatus being used for intracytoplasmic sperm injection application. The upper contact plate (5) has a central hole (7) through which a magnetizable rod (8) protrudes though and above the surface plane of the upper contact plate (5) and the fruste-conical edge (6). The magnetizable rod (8) is physically attached to the base plate (9) and both are currently magnetized as illustrated by the black filled in appearance of the magnetizable rod (8), which magnetically immobilizes the magnetized antibody (4), which is bound to antigens on the sperm (2). The recipient oocyte (1) with nucleus (3) rests atop the central hole (7) and fruste-conical edge (6) and is impaled by the magnetizable rod (8).

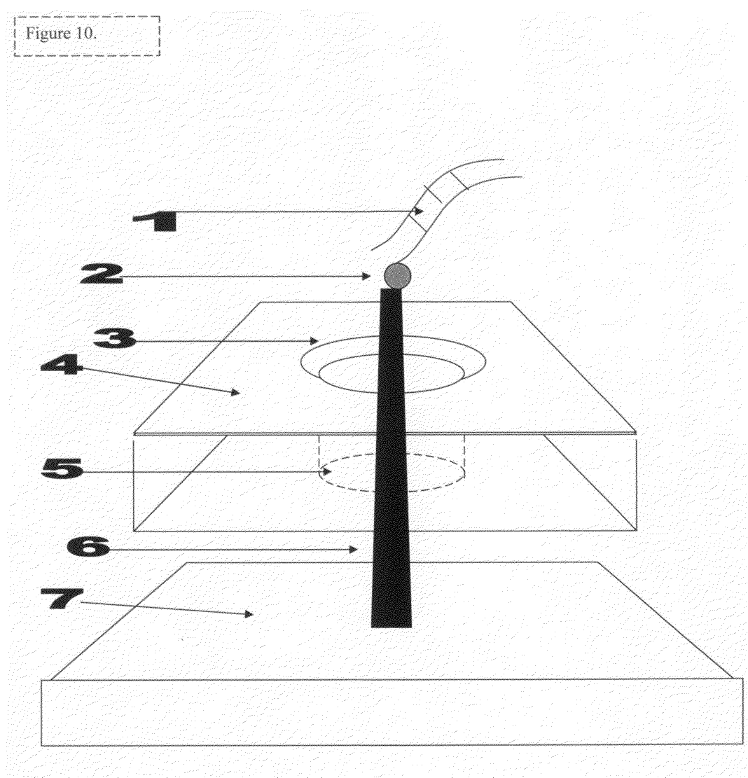

FIG. 10: Conceptual drawing of the smallest functional unit of the apparatus being prepared for transfection of genetic agents (1), whereby a magnetically responsive (2) is linked to the genetic agent (1) and is attracted to the magnetized rod (6) which is connected to the base plate (7) and protrudes through the fruste-conical edge (3) of the central hole (5), of the upper contact plate (4). Again, the magnetizable rod (6) and base plate (7) may be composed any suitable to allow a magnetizable surface which may be adjustable electronically.

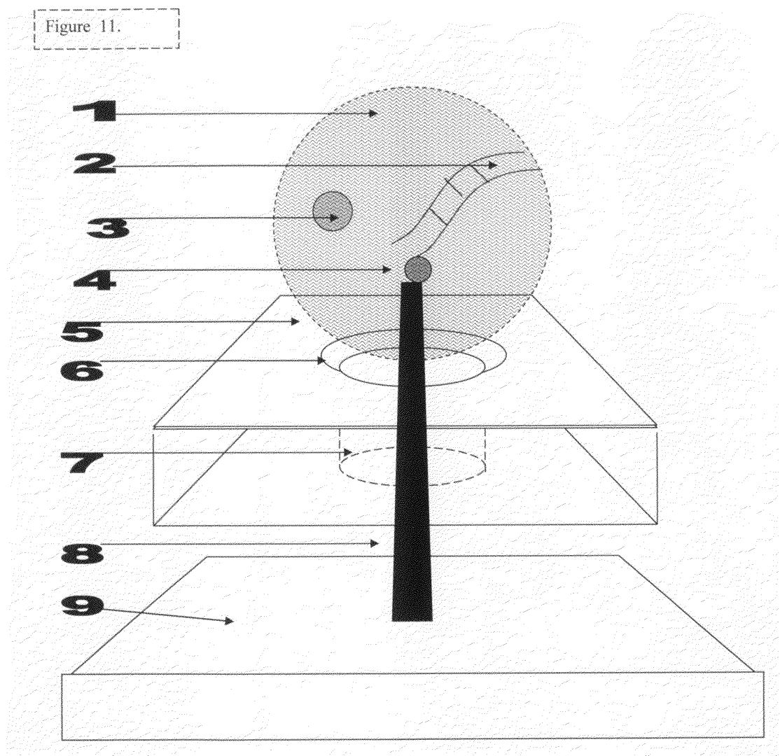

FIG. 11: Conceptual drawing of the smallest functional unit of the apparatus being used for transfection of genetic agents, whereby a magnetically responsive element (4) is linked to the genetic agent(s) (2) and introduced into a cell (1) with a nucleus (3). The genetic agent (2) is attracted to the magnetized rod (8) which is connected to the base plate (9) and protrudes through the fruste-conical edge (6) of the central hole (7), of the upper contact plate (5). Again, the magnetizable rod (8) and base plate (9) may be composed of any suitable material to allow a magnetizable surface which can be adjustable electronically.

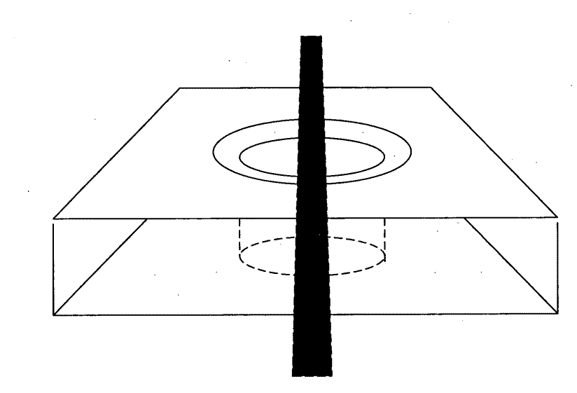

FIG. 12: Alternate conceptual drawing of the apparatus modified for transfection of genetic agents (1), but in this case three times the amount of transfection may occur as there are 3 magnetizable rods (6), whereby a magnetically responsive label (2) is linked to the genetic agents (1) and is attracted to the magnetized magnetizable rod (6) which is connected to the base plate (7) and protrudes through the, fruste-conical edge (4) of the central hole (5), of the upper contact plate (3). Again, the magnetizable rods (6) and base plate (7) may be composed any suitable material to allow a magnetizable surface which may be adjustable electronically.

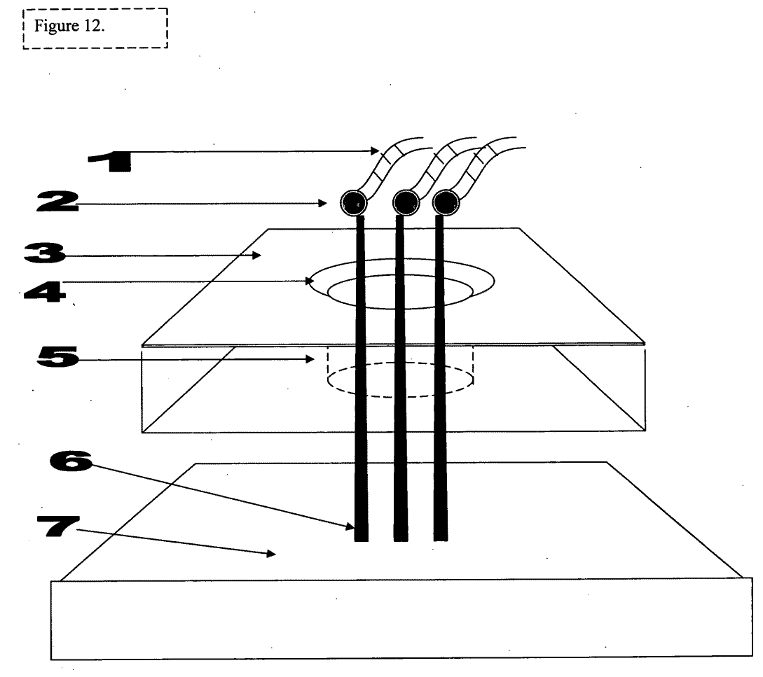

FIG. 13: 3-fold transfection of genetic agents (3) into the recipient cell (1) with intact nucleus (2). Three magnetizable rods (4) are pictured in this representation to show semi-quantitative transfection may be achieved by modifying the number of rods (4) protruding through the central hole (5) of the apparatus.

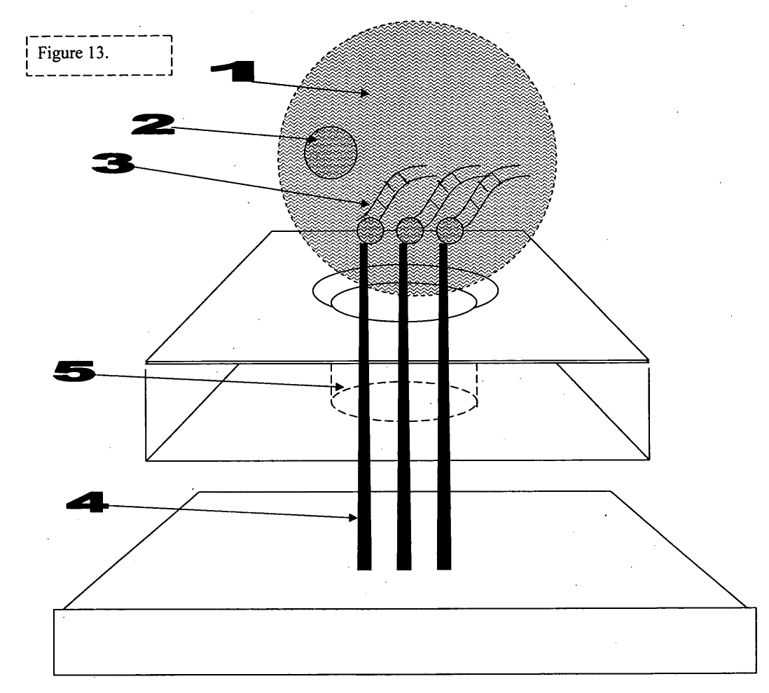

FIG. 14: Alternate arrangement of apparatus for genetic agents (1) transfer using magnetized antibodies (2) to attract bound genetic agents (1) to magnetized magnetizable rod (3).

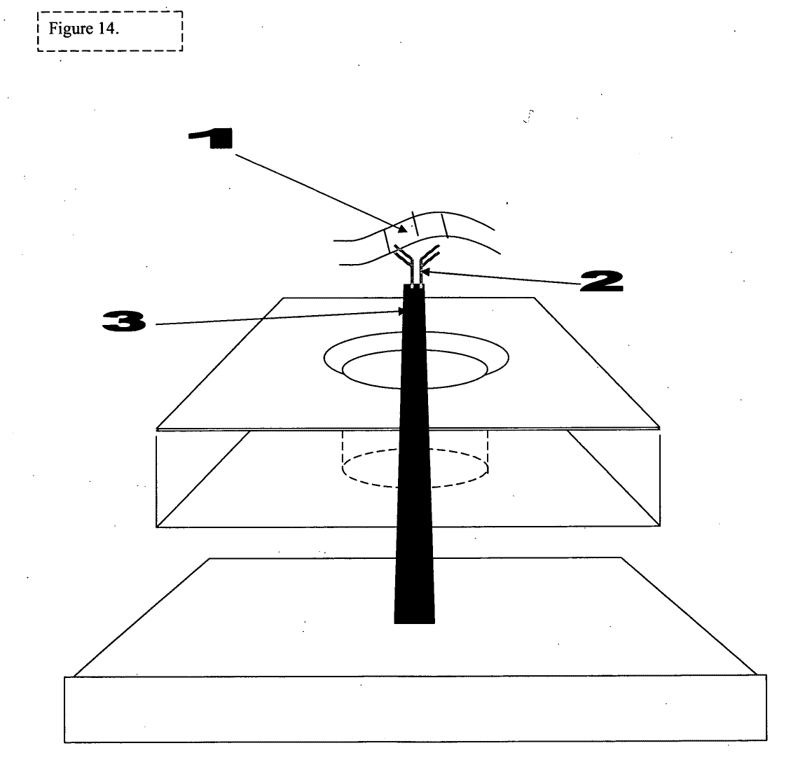

FIG. 15: An ideal three dimensional representation of the invention. Note magnetizable rods (3) fit through center (1) of upper contact plate (2). The lower base plate (4) is physically attached to the magnetizable rods (3) but a space between the upper contact plate (2) and lower lower base plate (4) exists to allow flow of tissue culture or buffer fluid between the plates.

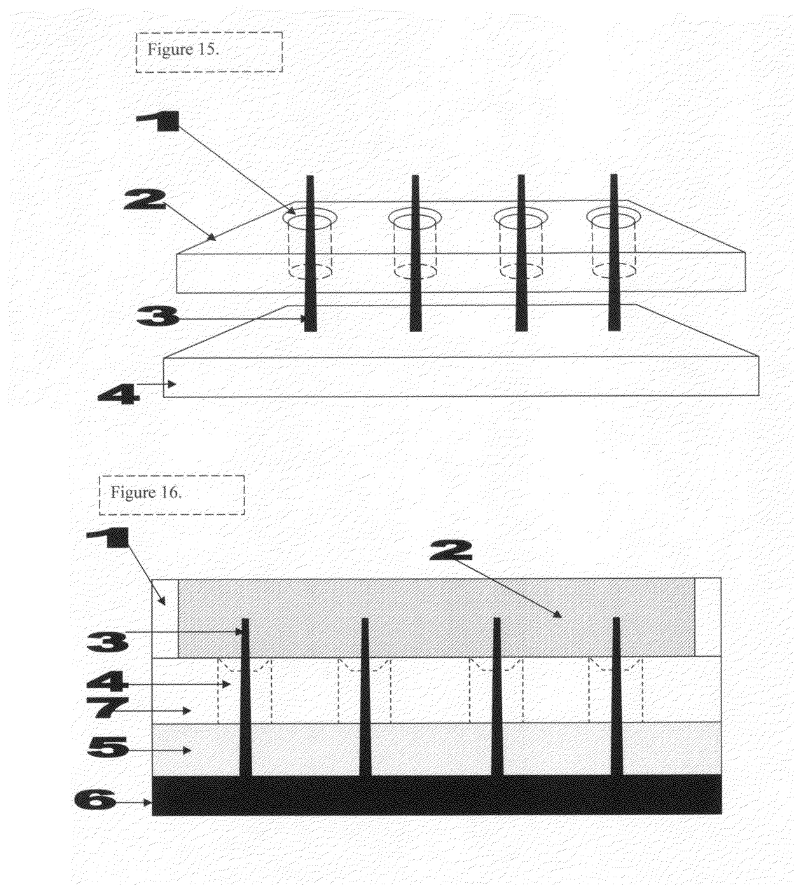

FIG. 16: Longitudinal cross section representation of device. The upper contact plate (7) is surrounded over the top surface with a ledge (1) to create a culture well space (2) to hold tissue culture media or buffer fluid. The magnetizable rods (3) are physically attached to the base plate (6) and protrude through the central hole (4). Note the space (5) between each plate (6 and 7) to allow flow through of cell culture media/buffer.

FIG. 17: Sagittal, cross-section view of device. The upper contact plate (7) is surrounded over the top surface with a ledge (2) to create a culture well space (1) to hold tissue culture media or buffer fluid. The magnetizable rods (4) protrude through the central hole (3) and is physically attached to the base plate (6). Note the flow through port (5) in the space (diagonal hatching) between each plate (6 and 7) to allow flow through of cell culture media/buffer.

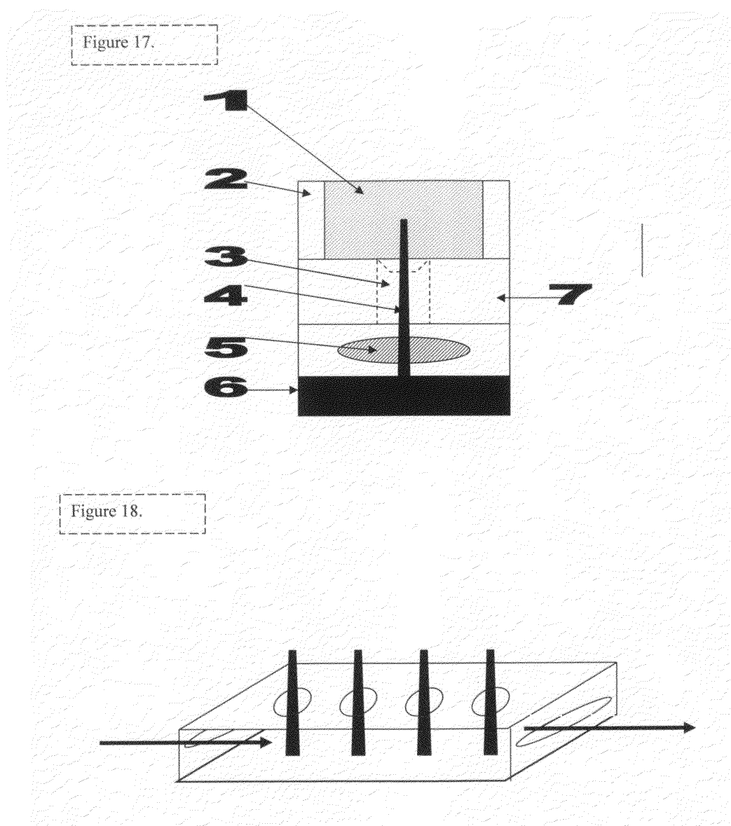

FIG. 18: Three dimensional view of invention with entry and exit flow through ports. Dark arrows signify direction of flow. Occlusion of the distal flow through port (right side of drawing) results in flow out of central holes of upper contact plate.

### FURTHER EMBODIMENTS

While at least one method of use of the apparatus has been described, a person of ordinary skill in the art can see that a variety of steps may be used for the method and apparatus, and it is possible to add or remove optional steps to the method detailed.

I claim:

1. A method of automated transfer of genetic agents in eukaryotic or prokaryotic cells at the microscopic and/or nanometric level, with the method comprising:
a. preparation of donor genetic material by:
i. removal of genetic agent by first coating it with magnetically responsive labels prior to introduction into the device
ii. introducing the donor cells to the first reaction chamber
ii. impaling the cell with an electronically magnetized rod with a vacuum at the base
iii. removing the rest of the donor cell by reversing the vacuum and washing away the cell into a waste chamber, but leaving the genetic agent magnetically bound to the rod
iv. alternatively using no donor cell but introducing into the first reaction chamber genetic agents with magnetically responsive labels
b. preparation of the recipient cells using the same device but in a separate second reaction chamber by:
i. removing redundant pre-existing genetic agents by coating it with 381 magnetically responsive labels prior to introduction into the device
ii. impaling the cell with an electronically magnetized rod with a vacuum at the base and
iv. removing the rest of the recipient cell by reversing the vacuum and leaving the genetic agent magnetically bound to the rod.
c. insertion of donor genetic agents into the recipient cells by:
i. introducing the treated recipient cells from 1b into the first reaction chamber of 1a.
i. impaling the recipient cell with the electronically magnetized rod holding the donor genetic agent by activating a vacuum at the base and
ii. releasing the genetic agent into the cytoplasm of the cell by electronically removing the magnetic field or reversing the polarity of the rod and finally
iii. releasing the cell by reversing the vacuum and allowing the newly transfected cell to roll free from the impaling rod into a collection chamber.

2. A device composed of:
a. contact plate with raised edges to allow containment of cell media/buffer
b. the same contact plate with a hole, with or without fruste-conical edges
c. a second base plate beneath the upper contact plate
d. a rod connected to the same base plate and protruding through the center of the contact plate hole past the plane of the contact plate
e. a space between the two plates to allow the flow of cell media/buffer which creates a vacuum affect through the contact plate holes in the direction of the base plate
f. an entry and exit flow port at the longitudinal ends of the plates communicating and regulating the flow of cell media/buffer
g. the same exit port may be occluded to cause positive pressure out the central holes of the upper contact plate away from the base plate and contact plate to push cells off the contact plate
h. the same rod and base plate is electronically magnetizable and adjustable in field strength, polarity and/or neutrality
i. the same rod may be coated with magnetic or paramagnetic genetic agents when magnetized
j. multiple reaction chambers with:
i. entry and exit channels leading to other reaction chambers or waste or collection chambers
ii. inter-chamber channels to cells and fluid to flow between the chambers
k. the same rod and hole which may be further arranged into an array of multiple rod hole units but upon the same base plate and within the same reaction chamber
l. fabrication being free of endotoxin
m. dimensions to allow the entire apparatus to be housed within a Petri or tissue culture dish or any suitable tissue culture container suitable for tissue culture
n. dimensions to allow visualization under a microscope

3. Alternately the method and device may be composed of or utilize:
a. a pleurality of rod and hole units to allow transfer and/or removal of genetic agents from multiple cells simultaneously
b. magnetized rods and/or base with a solid-state, pre-determined magnetic polarity and strength without the need or use of electricity
c. a pleurality of magnetizable rods within each hole to provide a semi-quantitative increase in transfer of genetic agents
d. standard antibodies (without any magnetization) recognizing and binding to the genetic agents and immobilized antibodies on the rod recognizing the constant portion of the antibodies bound to the genetic agents
e. translucent material to allow visualization under a standard or inverted microscope or scanning tunneling microscope or CCD or light sensing surface
f. flourochrome, fluorophore labels to visually detect genetic agents immobilized on the rods
g. glass, plastic, silicone or any suitable material to fulfill the functional criterion of the invention
i. manufacture by lithography, laser or chemical etching or any means necessary to fulfill functional criterion.
